(19) United States
(12) Patent Application Publication
(10) Pub. No.: US 2023/0405034 A1
(43) Pub. Date: Dec. 21, 2023

(54) FORMULATION OF IVERMECTIN IN SOFT GELATIN CAPSULES

(71) Applicant: PROCAPS S.A., BARRANQUILLA (CO)

(72) Inventors: Diego Rafael Monterroza Hernandez, Barranquilla (CO); José Alfredo Acosta Urrea, Barranquilla (CO); Rafael Fabián Domínguez Borja, Barranquilla (CO); Yudis Esther Gomez Tapia, Barranquilla (CO); Alfredo Berthel Beleño, Barranquilla (CO)

(21) Appl. No.: 18/029,370

(22) PCT Filed: Sep. 30, 2020
(86) PCT No.: PCT/IB2020/059182

§ 371 Date: Mar. 29, 2023

(51) Int. Cl. A61K 31/7048; A61K 47/14; A61K 47/10; A61K 47/22; A61K 9/48

(52) U.S. Cl. CPC A61K 31/7048; A61K 47/14; A61K 9/4825; A61K 47/22; A61K 47/10

(57) ABSTRACT

The present invention relates to a formulation of Ivermectin in soft capsules. In particular, the present invention relates to a formulation of Ivermectin in soft capsules which allows proper absorption in water, allowing the drug to be consumed at any time. Ivermectin, as an insoluble active ingredient, according to the invention, can be formulated in a liquid solution without compromising its stability and without creating impurities to form a suitable soft gelatin capsule. Therefore, the invention comprises a formulation comprising Ivermectin in a lipid medium which includes a surfactant in combination with an oily solvent, a co-surfactant and an oxidant, to form a soft capsule comprising a collagen coating, a plasticiser and gelatin. The field of the invention relates to improving the bioavailability of insoluble drugs such as Ivermectin for delivery in oral soft gelatin capsule forms.

### FIELD OF THE INVENTION

The present invention refers to a formulation of Ivermectin in soft capsules. More, particularly, the present invention refers to a formulation of Ivermectin in a soft capsule for oral administration, which allows correct absorption in aqueous gastrointestinal media, making it possible for the drug to be consumed at any time.

Ivermectin, as an insoluble active, according to the invention can be formulated in a liquid solution without compromising its stability and without the formation of impurities to form a suitable soft capsule. Therefore, the invention comprises a formulation that includes Ivermectin in a lipid medium that includes an oil, a surfactant in combination with a co-surfactant and an antioxidant agent, to form a soft capsule for oral administration.

The field of the invention is related to improving the bioavailability of insoluble drugs such as Ivermectin for delivery in oral soft capsule forms.

### BACKGROUND OF THE INVENTION

The active principle of Ivermectin is a drug that is complex and difficult to formulate. Such difficulty to formulate Ivermectin is due to its physicochemical characteristics such as degradation by hydrolysis, oxidation, acid medium, alkaline medium, light and temperature. The instability in turn makes it difficult to design a stable composition suitable for incorporating into a soft capsule.

Ivermectin is useful for controlling and treating a broad spectrum of infections caused by parasitic nematodes (roundworms) and arthropods (insects, ticks, and mites) that affect livestock and domestic animals.

The effects of these types of parasites can be serious. For example, ticks are responsible for the transmission and spread of many human and animal diseases throughout the world. The most economically important ticks include Boophilus, Rhipicephalus, Ixodes, Hyalomma, Amblyomma, and Dermacentor. They are vectors of bacterial, viral, rickettsian, and protozoal diseases, and cause tick paralysis and toxicosis. Even a single tick can cause paralysis by injecting its saliva into its host during the feeding process. Tick-borne diseases are generally transmitted by ticks from multiple hosts. These diseases, including babesiosis, anaplasmosis, theileriosis, and heartwater, are responsible for the death and/or debilitation of large numbers of domestic and food animals throughout the world. In many temperate countries, Ixodid ticks transmit the agent of a chronic debilitating disease, Lyme disease, from wildlife to man.

In addition to disease transmission, ticks are responsible for large economic losses in livestock production. Losses are attributable not only to death, but also to skin damage, growth loss, reduced milk production, and reduced meat quality.

Although Ivermectin is an FDA-approved broad-spectrum antiparasitic agent (Gonzalez Canga et al., 2008), researchers at the Biomedicine Discovery Institute at Monash University Melbourne have in recent years shown that Ivermectin possesses antiviral activity against a wide range of viruses (Gotz et al., 2016; Lundberg et al., 2013; Tay et al., 2013; Wagstaff et al., 2012) in vitro.

Originally identified as an inhibitor of interaction between the human immunodeficiency virus-1 (HW-1) protein mtefatase (IN) and the importin a/P1 heterodimer (IMP) responsible for the nuclear import of IN (Wagstaff et al., 2011), ivermectin has been confirmed that it inhibits IN nuclear import and HW-1 replication (Wagstaff et al., 2012). Other actions of ivermectin have been reported (Mastrangelo et al., 2012), but ivermectin has been shown to inhibit in a host nuclear import (p. (Kosyna et al., 2015; van der Watt et al., 2016)) and viruses, proteins, including simian virus SV40 large tumor antigen (T-ag) and virus non-structural protein 5 (DENV) (Wagstaff et al., 2012, Wagstaff et al., 2011). Importantly, it has been shown to limit infection by RNA viruses such as DENV 1-4 (Tay et al., 2013), West Nile Virus (Yang et al., 2020), Venezuelan Equine Encephalitis Virus (VEEV) (Lundberg et al., 2013), 2013) and influenza (Gotz et al., 2016), and this broad-spectrum activity is thought to be due to the reliance of many different RNA viruses on EVIPa/P1 during infection (Caly et al., 2012; Jans et al., 2019).

Similarly, ivermectin has been shown to be effective against pseudorabies DNA virus (PRV) both in vitro and in vivo, and ivermectin treatment increases survival in PRV-infected mice (Lv et al., 2018).). Ivermectin has also been shown to have antiviral activity against the causative agent of the current COVID-19 pandemic, SARS-CoV-2, which is a single-stranded, positive-sense RNA virus that is closely related to the severe acute respiratory coronavirus syndrome (SARS-CoV). Studies on SARS-CoV proteins have revealed a potential role of IMPa/pi during infection in signal-dependent nucleocytoplasmic closure of the SARS-CoV nucleocapsid protein (Rowland et al., 2005; Timani et al., 2005; Wulan et al., 2015), which can affect host cell division (Hiscox et al., 2001; Wurm et al., 2001). In addition, the SARS-CoV accessory protein ORF6 has been shown to antagonize the antiviral activity of the transcription factor STAT1 by sequestering IMPa/p1 in the rough ER/Golgi membrane (Fileman et al., 2007). Taken together, these reports suggested that Ivermectin's nuclear transport inhibitory activity may be effective against SARS-CoV-2 in vitro, causing an approximately 5,000-fold reduction in viral RNA after 48 hours.

According to said study, Ivermectin binds to the Impa/P1 heterodimer and destabilizes it, preventing Impa/pi from binding to the viral protein and preventing it from entering the nucleus. This likely results in reduced inhibition of antiviral responses, leading to a normal and more efficient antiviral response.

All this background investigations reveal new treatment scenarios associated with the use of Ivermectin as an effective drug in multiple pathologies.

Ivermectin is a slightly hygroscopic, white crystalline powder. It is practically insoluble in water. Therefore, it presents a significant challenge to improve the bioavailability of insoluble drugs such as Ivermectin for delivery in oral soft gelatin capsule forms.

It is known within the state of the art that the intake of drugs that incorporate Ivermectin in aqueous solutions should not be administered concomitantly with food, since its absorption and therefore its activity on the target of action is hindered.

The state of the art shows that Ivermectin is administered orally through aqueous solutions, tablets, and some creams are also used for topical treatments. However, the state of the art does not report compositions that include Ivermectin in soft gelatin capsules as disclosed in the present invention. The foregoing is due to the fact that the work of the formulator presents challenges in terms of dissolving the active ingredient to be incorporated into soft capsules while ensuring the stability and bioavailability of the active ingredient, precisely because of the aforementioned physicochemical characteristics.

Consequently, until today, there is no stable formulation that has been designed and developed that incorporates Ivermectin in a soft capsule and which shows differential and substantial characteristics compared to the previous state of the art.

The prior art reveals injectable liquid forms such as the one referenced in U.S. Pat. No. 5,788,978 by Passeron et al., where an injectable Ivermectin composition is defined that has a programmable release rate and that provides multiple active Ivermectin concentration peaks to produce a pulsed sequence of Ivermectin release in the blood of cattle and horses.

The composition comprises a 0.2%-10% w/w solution of Ivermectin in a solvent selected from propylene glycol and a mixture of glyceryl caprylate, caproate and caprate, such as glycerides of caproic, caprylic and capric acids in equal parts. The solution is used as a vehicle to suspend microspheres 100 nm-200 pm in diameter of a degradable polymer containing between 0.5% and 50% Ivermectin. The microspheres can be formed from polylactic acid, polyglycolic acid, or a polylactic-polyglycolic acid copolymer. The multi-pulse programmable release system can also be obtained with a biodegradable matrix selected from hardenable natural polymers, such as gelatin or albumin, as well as lactic and glycolic acid copolymers. Polymers can be subjected to a hardening process to increase resistance to biological agents, e.g. glutaraldehyde solution or alum, or by heating the proteins to the coagulation temperature.

In one embodiment, Ivermectin-loaded gelatin microspheres are treated in a 25% aqueous glutaraldehyde solution for 24 hours and then suspended in the solvent. Another embodiment includes a suspension of microspheres loaded with Ivermectin of 1:1 DL-lactic-glycolic copolymer. This ratio of monomers can be modified to improve erosion resistance. However, this liquid form cannot be encapsulated due to the excipients used, as well as surfactants and co-surfactants, where, as it is disclosed, it is poorly absorbed and therefore has low bioavailability in the digestive system.

U.S. Pat. No. 7,754,696 to Strobel Michael, illustrates a stable and pleasant solution of Ivermectin in water for mass medication of animals. The present formulation does not require the use of benzyl alcohol and is stable indefinitely in concentrated form and up to 30 days when mixed with water. Therefore, due to the poor shelf life, it is not useful for integration into a soft gelatin capsule formulation.

In view of what has been previously described, it is evident that the state of the art does not reveal compositions in soft gelatin capsules that incorporate Ivermectin. This is due to the fact that the active ingredient Ivermectin presents great formulation challenges as it is a complex molecule, whose physicochemical characteristics make it easily degradable by hydrolysis, oxidation, acid medium, alkaline medium, light and temperature. The aforementioned drawbacks are generated in terms of the stability of the product and solubility; with a related effect on bioavailability.

The present invention provides an optimal alternative for the administration of Ivermectin in a soft capsule that allows exact dosing, and adherence to treatment by the patient, since soft capsules are an ideal pharmaceutical form against masking the bitter tastes of medications that are administered, for example, in the form of drops.

Likewise, the present invention provides innovative and advantageous Ivermectin soft capsules that show rapid absorption and are easy to swallow and with good solubility of the active ingredient. The product of the invention shows good control regarding stability challenges and also provide increases in bioavailability.

### OBJECT OF THE INVENTION

Therefore, a first object of the present invention is to avoid the drawbacks of the prior art. Particularly, the main object of the present invention is to create a formulation of Ivermectin as an insoluble active in a liquid solution to form a soft capsule with effective bioavailability.

According to the present invention, the main object of the invention is to create a formulation of Ivermectin in a lipid medium in a soft capsule to improve release and solubility and therefore increase its bioavailability.

It is of paramount importance for formulation scientists to explore the potential of a self-emulsifying delivery system by combining appropriate excipients based on critical parameters such as surfactant concentration, oil/surfactant ratio, fine oil droplet size and compatibility, which allows it to be administered as a unitary form for oral administration.

Another important object is the mechanisms involved to improve the solubility and bioavailability of the active without compromising stability and therefore the creation of impurities.

Another also important object is to solubilize without compromising stability without the creation of impurities.

The present invention meets these needs and provides other related advantages. The novel features which are considered to be the basis of the invention are set forth in particular in the appended claims and the additional advantages thereof, will be better understood from the following detailed description with the preferred embodiments.

### DETAILED DESCRIPTION OF THE INVENTION

The present invention refers to a formulation of Ivermectin incorporated into soft capsules. Particularly, the present invention refers to a formulation of Ivermectin as an insoluble active in a liquid solution with optimal stability and without the formation or presence of impurities to incorporate and make an adequate soft capsule.

The invention relates to improving the bioavailability of insoluble drugs such as Ivermectin for delivery in oral softgel forms.

Particularly, the active principle of Ivermectin is complicated and difficult to formulate, because it is affected by both the internal and external environment, such as light, heat, humidity and in the way that gastrointestinal acids affect it, since it is a molecule that is insoluble in water.

For this purpose, the formulation of the invention is designed by formulating it as a lipid-based drug delivery system whose excipients control the formation of impurities and their choice defines their release and solubility for effective bioavailability. Consequently, the invention was developed from a system that uses a micro emulsion achieved by chemical means.

A self-emulsifying system consisting of an oily phase (oil), a surfactant and a co-surfactant is selected to influence the solubility of the poorly soluble active and promote its bioavailability.

The formulation according to the present invention comprises the use of medium chain triglycerides in a proportion of between 1 to 10 mg per mL. In this regard, the inventors have found that medium chain triglycerides are excellent solubilizers for the active ingredient and generate, together with the surfactant/co-surfactant, a self-emulsifying and compatible formulation for an suitable encapsulation.

The oily phase comprises about 95% medium chain triglycerides and is selected for its role in the solubilization of Ivermectin and its high polarity and high release.

Based on the invention, it has been found that medium-chain fatty acid triglycerides provide solubility in water faster than long-chain fatty acid triglycerides and this faster solubility is measured by a laboratory-level assay (EHM) by hydrolysis using NaOH. The assay value for hydrolysis of the medium chain fatty acid triglycerides is 14.9 ml NaOH as compared to other triglycerides that require less (higher titrant consumption=higher degree of hydrolysis/lipolysis).

This medium chain triglyceride (MCT) can be chosen from caprylic/capric/lauric fatty acids. The oil phase important ingredients in the formulation are glycerol fatty acid esters containing 8 to 12 carbon atoms and are selected for their high solvent capacity of Ivermectin and this capacity is mainly decided by the effective concentration of ester groups and for being less prone to oxidation due to the absence of unsaturated acids.

The solubility of Ivermectin in the oily phase in the presence of MCT is increased by incorporating a lipophilic nonionic surfactant, which also behaves as a bioavailability enhancer in oral formulations.

The addition of a co-surfactant in the formulation favors the stability of the microemulsion and reduces the interfacial tension to a very small or negative value, to help the dispersion and absorption of Ivermectin in the self-emulsifying system.

Additionally, the incorporation of an antioxidant to the formulation proposed in the present invention prevents or controls the oxidation of the components present, especially the oils whose physicochemical characteristics make them more susceptible to degradation.

The formulation according to the present invention comprises a proportion of Ivermectin in an amount of between 0.5 to 2.0% by weight of the formula; an amount of oily solvents based on medium chain triglycerides containing 8 to 12 carbon atoms in an amount between 60 to 80%; a surfactant such as linoleoyl polyoxyl-6 glycerides in a proportion of 10 to 20%; a co-surfactant such as propylene glycol monocaprylate (>90% monoesters) in a quantity of 10 to 20% and in a preferred embodiment may comprise an antioxidant in a quantity from 0.1 to 0.9% to complete the formulation.

Particularly, the formulation comprises a liquid solution incorporated into a soft capsule and said formulation comprises by weight Ivermectin in an amount of 1.2%, oily solvents based on medium chain triglycerides in an amount of 70%, a surfactant in an amount of 14%, a >90% monoester co-surfactant in a quantity of 14% and a proportion of 0.2% of an antioxidant.

The shell can be formed from gelatin in combination with 20% by weight of collagen.

Among the oily solvents selected for the composition of the present invention, it can be selected from canola oil, corn oil, cottonseed oil, sesame oil and soybean oil. It can also be based on Medium Chain Triglycerides (MCT) such as CAPTEX™ 300, Miglyol™ 810 (Caprylic/Capric Triglyceride) and Miglyol™ 812 (extracts from endosperms of palm oil and or coconut plants).

Surfactants selected for the present invention comprise nonionic solubilizing and emulsifying agents such as polyoxyl-32 lauroyl glyceride (Gelucire™ 44/14), caprylocaproyl macrogolglyceride (Labrasol™) and polyoxyl-6 linoleoyl glyceride (Labrafil™ M 2125 CS and Labrafil™ M 1944 CS).

Suitable co-surfactants are selected from Capryol 90 (Propylene Glycol Monocaprylate), Lauroglycol 90 (Propylene glycol monolaurate), CAPTEX™ 200 (Propylene Glycol Dicaprylate/Dicaprate), Miglyol™ 840 (Propylene Glycol Dicaprylate/Dicaprate), Labrafac™ lipophile WL 1349 (medium-chain triglycerides of caprylic (C₈) and capric (C₁₀) acids), and Span™ 80 (Sorbitan monooleate).

The antioxidant is selected from the group consisting of butylhydroxyanisole (BHA), butylhydroxytoluene (BHT), propylgallate, sesamol, ascorbic acid, ascorbyl palmitate, malic acid, sodium ascorbate, sodium metabisulfite, tocopherol, and DL-alphatocopherol.

For its part, the liquid solution is incorporated into a soft capsule that includes a coating that comprises a film-forming material and at least one plasticizer.

The shell containing the composition incorporates a film-forming material selected from the group consisting of gelatin of animal or vegetable origin in combination with glycerin sorbitol-sorbitan and propylene glycol plasticizers. Preferably, the shell can optionally comprise up to 20% by weight of collagen.

In relation to digestibility, the liquid solution comprises a self-emulsifying composition and the oily phase that integrates it, allows through the glycerol medium chain fatty acids (MCFAs=C₈:50-80%−C₁₀:20-50%−C₁₂:3%=chain with a length of 8 to 12 C atoms), generating a greater digestibility to guarantee adequate bioaccessibility of the bioactive compound.

Regarding hydrolysis, medium chain triglycerides are fatty acid esters of glycerol and they are rapidly hydrolyzed.

Due to their smaller molar mass (shorter chain lengths), they do not require the formation of chylomicrons for their uptake and transport (EHM=Estimated Hydrolysis Maximum: 14.95 ml of NaOH consumed in vitro in the medium chain triglyceride lipolysis assay.

It was also found that, in relation to lipolysis, the half-life in seconds of medium chain triglycerides is 3349 compared to others such as 715 for mono-diglycerides of medium chain fatty acids (mainly capric).

Regarding digestion and absorption, M-medium-chain triglycerides are fatty acid esters of glycerol and are digested more rapidly and the released medium-chain fatty acids (MCFAs) are absorbed directly into the bloodstream through the portal system of the arteries and intestinal microvilli because they are soluble in water. Of note, medium chain triglycerides do not stimulate gastrointestinal hormones and therefore do not require bile or pancreatic enzyme, nor they require micelle formation prior to absorption and are absorbed into the albumin-bound portal circulation. In particular, it does not require carnitine as transporter to the mitochondria and it is stored little in adipose tissues. The plasma half-life is approximately 17 minutes, compared to 33 minutes for long-chain triglycerides and other types of glycerides.

The solubility of Ivermectin in the oily phase can be increased by incorporating a lipophilic nonionic surfactant that also behaves as a bioavailability enhancer for oral formulations of the self-emulsifying drug delivery system and a self-emulsifying drug delivery system. emulsifier, where this constituent surfactant of the composition is linoleoyl polyoxyl-6 glyceride.

Digestion of the linoleoyl polyoxyl-6 glyceride surfactant in vivo leads to dynamic changes in the composition of gastrointestinal fluids, thus increasing the solubilization capacity of the active ingredient.

The motility of the stomach and intestine provide the necessary agitation for autoemulsification; An example of this are the factors that control the in vivo performance of self-emulsifying systems that include the ability to form very small oil droplets (<5 microns) and the polarity of these droplets that promote rapid release of the active ingredient in the aqueous phase.

The addition of a co-surfactant propylene glycol monocaprylate (>90% monoesters) formulation comprises a C₈ fatty acid monoester that maintains the HLB value of the surfactant, favoring the stability of the microemulsion.

This co-surfactant acts as a second surfactant reducing the interfacial tension to a very small or negative value and improves the incorporation of Ivermectin for better dispersibility and absorption in the formulation.

Therefore, based on the invention, the formulation of the self-emulsifying invention is designed with the proper selection of oil, surfactants and co-surfactant, to influence the absorption of the poorly soluble active in water and facilitate bioavailability and oral bioactivity.

It should be taken into account that the non-aqueous solution formulation of Ivermectin should be taken before food.

The lipid medium proposed according to the present invention facilitates the absorption of water-insoluble active ingredients and allows the drug to be consumed at any time.

Only some preferred embodiments of the invention have been illustrated by way of example. In this regard, it will be appreciated that the formulation of Ivermectin in soft capsules, as well as the configurative arrangements can be chosen from a plurality of alternatives without departing from the spirit of the invention according to the following claims.

1-9. (canceled)

10. A formulation of Ivermectin suitable for incorporation into softgel capsules which formulation comprises:
(a) Ivermectin in an amount between 0.5 to 2.0% by weight;
(b) 60 to 80% by weight of an oily solvent;
(c) 10 to 20% by weight of a surfactant;
(d) 10 to 20% by weight of a co-surfactant; and
(e) 0.1 to 0.9% by weight of an antioxidant; and wherein said formulation exhibits enhanced bioavailability upon oral administration.

11. The formulation of claim 10, wherein said oily solvent is selected from the group consisting of canola oil, corn oil, cottonseed oil, sesame oil, soybean oil and medium chain triglycerides.

12. The formulation of claim 11, wherein said medium chain triglycerides is selected from the group consisting of Caprylic/Capric Triglyceride and extracts from endosperms of palm oil and or coconut plants.

13. The formulation of claim 10, wherein said surfactant is selected from the group consisting of: polyoxyl-32 lauroyl glycerides, caprylocaproyl macrogolglyceride and polyoxyl-6 linoleoyl glyceride.

14. The formulation of claim 10, wherein said co-surfactant is selected from the group consisting of: propylene glycol monocaprylate, propylene glycol monolaurate, propylene glycol dicaprylate/dicaprate, medium-chain triglycerides of caprylic (C₈) and capric (C₁₀) acids), and sorbitan monooleate.

15. The formulation of claim 10, wherein said antioxidant is selected from the group consisting of: butylhydroxyanisole (BHA), butylhydroxytoluene (BHT), propylgallate, sesamol, ascorbic acid, ascorbyl palmitate, malic acid, sodium ascorbate, sodium metabisulfite, tocopherol and DL-α-tocopherol.

16. The formulation of claim 13, wherein said surfactant is linoleoyl polyoxyl-6 glyceride

17. The formulation of claim 14, wherein said co-surfactant is propylene glycol monocaprylate.

18. The formulation of claim 14, wherein said co-surfactant is propylene glycol dicaprylate/dicaprate.

19. The formulation of claim 15, wherein said antioxidant is butylhydroxyanisole.

20. The formulation of claim 15, wherein said antioxidant is DL-α-tocopherol.

21. A softgel capsule having incorporated therein an Ivermectin formulation which comprises:
(a) Ivermectin in an amount between 0.5 to 2.0% by weight;
(b) 60 to 80% by weight of an oily solvent;
(c) 10 to 20% by weight of a surfactant;
(d) 10 to 20% by weight of a co-surfactant; and
(e) 0.1 to 0.9% by weight of an antioxidant.

22. The softgel capsule of claim 21, wherein said capsule is made from gelatin.

23. The softgel capsule of claim 22, wherein said gelatin is of animal or vegetable origin.

24. The softgel capsule of claim 23, wherein said gelatin further includes a plasticizer.

25. The softgel capsule of claim 24, wherein said gelatin plasticizer is glycerin.

26. The softgel capsule of claim 24, wherein said gelatin plasticizer is sorbitol.

27. The softgel capsule of claim 23, wherein said gelatin further includes collagen.

28. The softgel capsule of claim 27, wherein said collagen is present at up to 20% by weight.

29. The softgel capsule of claim 27, further including a plasticizer.
